(19) United States
(12) Patent Application Publication
(10) Pub. No.: US 2016/0143613 A1
(43) Pub. Date: May 26, 2016

(54) METHOD, APPARATUS, AND ARTICLE FOR ULTRASOUND BLOOD FLOW MEASUREMENT

(71) Applicant: GENERAL ELECTRIC COMPANY, SCHENECTADY, NY (US)

(72) Inventors: ERIK NORMANN STEEN, MOSS (NO); STEN ROAR SNARE, OSLO (NO)

(73) Assignee: GENERAL ELECTRIC COMPANY, SCHENECTADY, NY (US)

(21) Appl. No.: 14/549,938

(22) Filed: Nov. 21, 2014

(51) Int. Cl. A61B 8/06; A61B 8/00; A61B 8/08

(52) U.S. Cl. CPC A61B 8/06; A61B 8/488; A61B 8/5246; A61B 8/4444; A61B 8/4405

(57) ABSTRACT

A method includes obtaining first and second ultrasound image data and

computing a coarse transverse flow field by applying an optical flow technique to compare the second ultrasound image data to the first ultrasound image data at a coarse scale. The method further includes computing a fine transverse flow field by applying optical flow technique to compare the second ultrasound image data to the first ultrasound image data at a scale that is finer than the coarse scale and superimposing the fine transverse flow field onto the coarse transverse flow field to form a first combined flow field.

### BACKGROUND

1. Technical Field

Embodiments of the invention relate generally to ultrasound imaging. Particular embodiments relate to techniques for ultrasound measurement of fluid velocity.

2. Discussion of Art

Ultrasound often is used for imaging internal structures in medical settings. In addition to imaging internal structures, it is often desirable to estimate the velocity of fluid within such structures, e.g., blood flow within an artery or vein, in case the ultrasound image might be used in diagnosing disorders such as arteriosclerosis or thrombosis. To date, however, ultrasound blood flow imaging has been limited to measuring displacements/velocities along the ultrasound beam, using either Doppler or autocorrelation techniques, and displaying only velocity components towards or away from the probe. For optimal diagnostic imaging, at least six dimensions of information are preferable (three velocity components, and three position components). Doppler scanning at best provides only four dimensions (position, plus an axial or radial component of velocity). As a result, there has been a need for skilled manipulation of an ultrasound probe, in order to correct for scanning angle and approximate measurement of complex flows.

It is also generally desirable when using an imaging system to have the capability of “real time” display, i.e., what an operator sees displayed on a screen corresponds closely to the structures toward which the imaging system is concurrently directed. For example, if using ultrasound to image blood flow in a fetus, it is highly desirable that when the ultrasound wand is directed toward the fetal heart, the imaging apparatus displays blood velocities in the heart. This type of real time information is extremely helpful for providing context to inform diagnosis, e.g., whether maternal or fetal positioning might be affecting blood flow.

To estimate blood flow in two dimensions, it is possible to apply pattern block matching schemes to ensemble echo data, limiting the tracking to stay within a group of parallel receive beams. The temporal distance between matching images then is the inverse of the pulse repetition frequency (PRF) of the system. By setting PRF sufficiently large, ensemble samples can be taken close enough in time so that the image speckle produced by flowing blood does not decorrelate between samples. Transverse flow velocity then can be estimated by tracking the inter-sample movement of speckle patterns across the beams. Block matching schemes, however, require computationally demanding steps of pattern identification and search, frequently incorporate “good enough” matching factors that may not in fact turn out to be good enough, and when they fail, the errors typically are found as outliers that can obscure, detract from, or discredit otherwise meaningful results.

Additionally, basic block matching schemes only can detect integer pixel movements. In case a speckle pattern moves more or less than a whole number of pixels across a pair of samples, a null or out-of-range result can be returned. Accordingly, systems that attempt to enhance inter-sample fidelity of speckle patterns, by using increasingly high-PRF, can have trouble resolving slow or even typical fluid velocities in which the speckle patterns do not move fast enough to cross beams between the closely-spaced samples.

One potential solution is to up-sample in space, i.e., capture an image with more and smaller pixels. As will be appreciated, however, additional pixels demand additional processor time, and due to the nature of block matching algorithms, the processor load scales faster than the image size. These considerations make it hard to implement reliable block matching schemes for real-time visualization of normal or sub-normal blood velocities based on speckle pattern matching.

In view of the above, it is desirable to provide apparatus and methods for real time ultrasound measurement of transverse blood velocities, without relying on speckle pattern matching. It is also desirable to provide apparatus and methods that generally improve real time ultrasound measurement of fluid velocities transverse an ultrasound beam pattern.

### BRIEF DESCRIPTION

Embodiments of the invention implement a method that includes obtaining first and second ensemble ultrasound image data; computing a coarse transverse flow field by applying optical flow technique to compare the second ensemble ultrasound image data to the first ensemble ultrasound image data at a coarse scale; computing a fine transverse flow field by applying optical flow technique to compare the second ultrasound image data to the first ultrasound image data at a scale that is finer than the coarse scale; and superimposing the fine transverse flow field onto the coarse transverse flow field to form a first combined flow field.

Other embodiments provide a display processing unit operatively connected to receive ultrasound image data from an ultrasound probe, and configured to obtain first and second ultrasound image data by scanning a target object, compute a coarse transverse flow field by applying optical flow technique to compare the second ultrasound image data to the first ultrasound image data at a coarse scale, compute a fine transverse flow field by applying optical flow technique to compare the second ultrasound image data to the first ultrasound image data at a scale that is finer than the coarse scale, and superimpose the fine transverse flow field onto the coarse transverse flow field.

Yet other embodiments provide an article, which is non-transitory computer readable media encoded with a velocity vector field visualization produced by a process that includes obtaining first and second ultrasound image data, computing a coarse transverse flow field by applying optical flow technique to compare the second ultrasound image data to the first ultrasound image data at a coarse scale, computing a fine transverse flow field by applying optical flow technique to compare the second ultrasound image data to the first ultrasound image data at a scale that is finer than the coarse scale, and superimposing the fine flow transverse field onto the coarse transverse flow field.

### DRAWINGS

The present invention will be better understood from reading the following description of non-limiting embodiments, with reference to the attached drawings, wherein below:

FIG. 1 is a perspective view schematically showing an ultrasound diagnostic apparatus for implementing embodiments of the present invention.

FIG. 2 is a schematic diagram that depicts a sequence of Doppler ultrasound pulses and receive scans for ensemble tracking according to an embodiment of the present invention.

FIG. 3 is a schematic diagram depicting an optical pyramid comparison of sequential Doppler ultrasound images for use in optical flow technique, according to an embodiment of the present invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts, without duplicative description. Although exemplary embodiments of the present invention are described with respect to ultrasound imaging of blood flow for medical purposes, embodiments of the invention also are applicable for use in ultrasound measurement of transverse fluid velocities, generally. Therefore, embodiments of the invention advantageously provide for ultrasound imaging of a 2D/3D flow velocity field in hard real-time (i.e., imaging updated after each ultrasound pulse within a scan) or real time (i.e., imaging updated after each ultrasound scan sequence) so that the flow velocity image can be displayed to a user synchronously with operation of an ultrasound instrument.

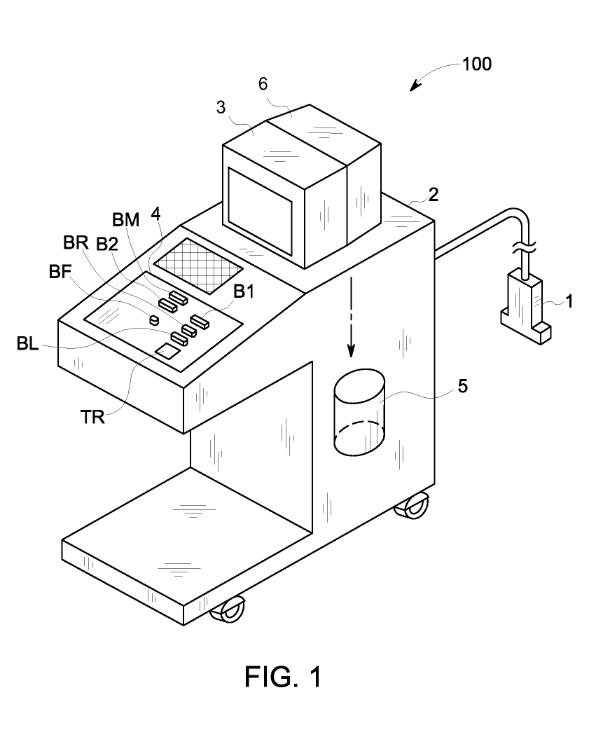

Referring now to FIG. 1, an ultrasound diagnostic apparatus 100 that is configured for use with embodiments of the present invention is depicted. The ultrasound diagnostic apparatus 100 includes an ultrasonic probe 1 which transmits an ultrasonic pulse into a subject (the body of a patient) and receives an ultrasonic echo from within the subject. The apparatus 100 further includes an ultrasound diagnostic apparatus body 2, which generates an ultrasound image on the basis of the ultrasonic echo, and a monitor 3 that displays the ultrasound image. The ultrasound diagnostic apparatus body 2 is provided with an operation panel 4, which accepts the operator's instructions, and a storage device 5 for storing ultrasound images and values of each item measured from the ultrasound images. The storage device 5 is, for example, a hard drive. The apparatus 100 further includes a display processing unit 6. An example of actual hardware of the display processing unit 6 may conceivably comprise a CPU for processing, ROM to store a program of the above-described configuration, and RAM, which can be used as a working area to rewritably stores various data. All components may be connected by a bus.

In response to operator instructions at the operation console 4, the ultrasonic probe 1 is caused to transmit ultrasound pulse sequences and to receive echo series according to conventional modes of operation and image pickup conditions. Specific image pickup conditions include, for example, the type of ultrasonic probe used for image pickup, e.g., linear scanning, sector scanning, mechanical scanning, or convex scanning, an ultrasonic pulse repetition frequency, the dynamic range of received echo intensity, the gain defining the relative amplification of received echo intensity, the velocity scale range defining the range of spectral display according to the velocity of blood flow, the gray map used as the conversion map for displaying received echo signals in different shades of gray, the color map for use as the conversion map for color displaying, the color area position defining the position of the interest area cursor for color Doppler signal detection, the sample volume position defining the detecting position for acquiring Doppler blood current signals, and the time scale range defining the time width per unit pixel. All these conditions are monitored, established, and computed by the display processing unit 6.

In the ultrasound diagnostic apparatus 100, the ultrasonic probe 1 emits one or more ultrasonic pulses scheduled at a pulse repetition frequency (“PRF”) and recovers ultrasonic echo signals that are returned from the subject to a plurality of two-dimensionally distributed sampling points. The ultrasonic probe 1 transduces the ultrasonic echo signals into digital data that is sent to the display processing unit 6. The display processing unit 6 then generates ultrasound images on the basis of the digital data provided from the ultrasonic probe 1, and sends the ultrasound images to the monitor or other display 3.

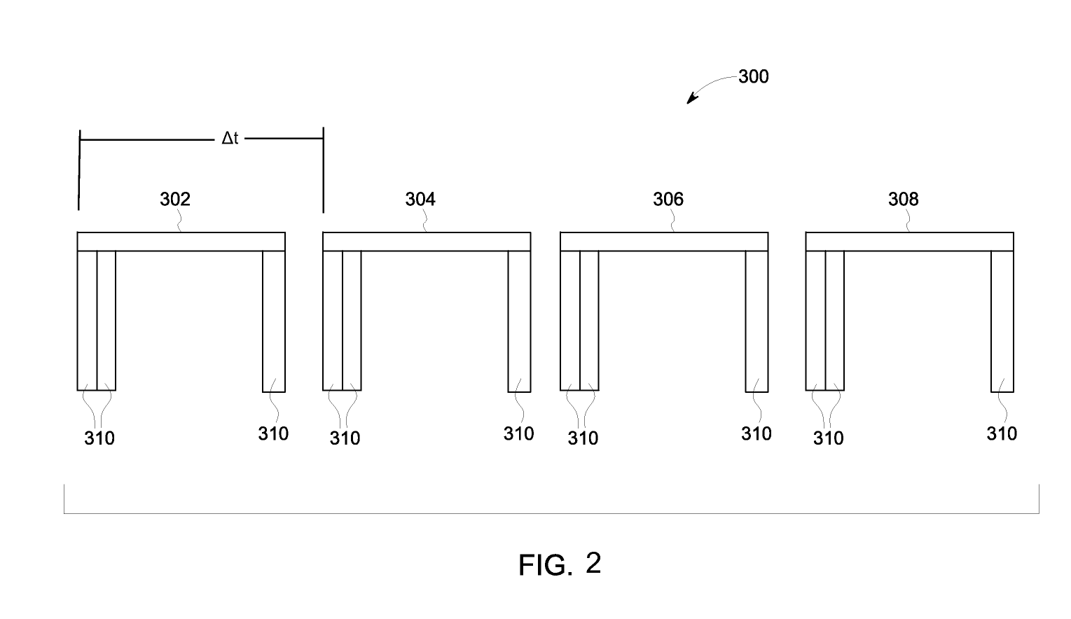

The above-described apparatus 100 is configured for use with embodiments of the present invention. More specifically, in an embodiment, as shown schematically in FIG. 2, the ultrasound probe 1 transmits and receives a sequence 300 of ultrasound pulses or packets, then samples the echoes in parallel beams (“MLA beams”) 310 when in ensemble tracking mode. For example, the ultrasound packet sequence 300 may include a first packet 302, a second packet 304, a third packet 306, and a fourth packet 308. Each MLA beam 310 then receives a set of ultrasound image data. Together, the packet sequence 300 results in an ensemble of Doppler ultrasound image data.

As mentioned, ensemble tracking enables Doppler measurement of along-beam (axial, or radial) flow velocity. Ensemble tracking also generates speckle patterns (based both on differing Doppler shifts, as well as random variation, among adjacent portions of the flowing fluid). Block matching of speckle patterns, as mentioned above, can be used for estimating transverse flow velocity based on integral pixel movement. In case movement is not an integral number of pixels, however, block matching can produce a null result. Accordingly, it has been known to augment (up-sample) pixel resolution, in order to avert the problem of non-integral pixel movement. Pattern matching computation load, however, scales faster than the total number of pixels and up-sampled block matching has not proven feasible for hard real time, real time, or near real time assessment of transverse flow velocities.

By contrast, embodiments of the invention provide for hard real time, real time, or near real time ultrasound measurement of fluid velocities transverse an ultrasound beam pattern. Generally, apparatus and methods according to the invention implement fluid velocity measurement using a pyramidal optical flow approach, rather than block matching. Optical flow performs best on sub-pixel motion, but it can also be applied to larger displacements by applying it in an image pyramid in which adjacent pixels are averaged together, for example by a Gaussian average. The computation time is a fraction of contour matching or speckle pattern matching, and there is less need for up-sampling.

Embodiments of the invention are intended to apply pyramidal optical flow techniques, in order to generate a six dimensional flow vector field (i.e. three dimensions of position and three dimensions of velocity) from ensemble data. Optical flow is a technique for generating a vector field from time spaced image samples, and has been considered particularly suitable for estimating vector fields from image samples that exhibit only sporadic change or movement. Optical flow techniques, however, are based on Taylor series expansion of a brightness constancy constraint:

$$I ( x , y , t ) = I ( x + Δ x , y\_Δ y , t + Δ t ) \approx I ( x , y , t ) + \frac{\partial I}{\partial x} Δ x + \frac{\partial I}{\partial y} Δ y + \frac{\partial I}{\partial t} Δ t .$$

Whereas the brightness constancy constraint presumes that image intensity (brightness) will be uniform within a “small” spatial and temporal neighborhood, e.g., among adjacent pixels across consecutive images, the Taylor series expansion used for optical flow presumes that brightness will be conserved smoothly among space-and-consecutive pixels.

For various reasons, ultrasound images typically violate the brightness constancy constraint that is an essential presumption of optical flow techniques. In particular, optical flow is applicable mainly to sub-pixel flow velocities across an image (i.e., the fluid particles move less than one pixel between adjacent images). Therefore, optical flow techniques have not found wide applicability in ultrasound imaging, which typically has strived for enhanced clarity and resolution (ever-smaller pixellations). Ensemble tracking, however, is a particular type of ultrasound measurement that is done for obtaining a Doppler shift of fluid moving toward or away from the ultrasound sensor. In ensemble tracking, multiple transmits 302, 304, 306, 308 are made, and multiple samples 310 are taken, in a same direction using an array of receive beams. The temporal distance between ensemble samples (Δt) is only 1/PRF (where PRF is the sampling pulse repetition frequency).

An aspect of the invention is the discovery that, for sufficiently high PRF, Δt could be sufficiently small so as to surprisingly not violate the brightness constancy constraint. If the brightness constancy constraint was preserved in ensemble tracking data, then optical flow techniques could become feasible. Thus, certain aspects of the invention relate to use of optical flow techniques for analyzing image data produced from ensemble tracking. Optical flow in regular ultrasound is very difficult on rapidly moving objects, such as the heart, because the subpixel movement assumption normally is severely violated. By using a Doppler acquisition, the movement between consecutive temporal samples is so small that the motion can be assumed to be subpixel or near-subpixel (resolvable by using a pyramid with few levels). This enables the use of optical flow. Use of optical flow has potentially large benefits compared to regular block matching.

In a Cartesian scan-space using optical flow, however, a maximum measurable transverse velocity is:

\vec{ν}ₘₐₓ=PRF*Δ\vec{r};

Δ\vec{r}=Δx,Δy.

Thus, for example, using optical flow with a PRF of 6 kHz on an image of (140,300) pixel across a region of 3 cm×3.5 cm, then ~νₘₐₓ=(1.29, 0.70) m/s. In medical imaging, pathology may induce blood flows at up to several m/s (e.g., up to 4 m/s), which using ensemble sample data would result in multipixel movements that again would violate the brightness constancy constraint.

Similarly, in a polar space as can be useful for adult cardiac exams, the maximum measurable velocity gets spatially dependent. Close to the probe, the beams have virtually no spacing, making the maximum measurable lateral velocity negligible. In radial and angular velocity, the maximum velocities approach:

\vec{ν}ₘₐₓ=(\vec{ν}_{rad},\vec{ν}_{θ})PRF*(Δr,Δθ)

For the same image size as above, (140; 300)pix, imaging depth of 16 cm, and a sector width of ⅓rad=75 deg a PRF of 4 kHz would yield

(\vec{ν}_{rad,max},\vec{ν}_{θ,max})=(2.13 m/s; 37.14 rad/s).

Using a small angle approximation, the velocity component normal to the beam can be found by:

\vec{ν}_{rad,max}≈r*\vec{ν}_{θ,max}

This equation yields a detectable cross-beam velocity ranging from 0 m/s at zero depth to 5.94 m/s at a maximum investigation depth of 16 cm. In a focal region around 7 cm, the maximum velocity would be 2.6 m/s.

Other embodiments of the invention relate to applying optical flow to ensemble data in an iterative image pyramid process. According to such an image pyramid process, optical flow techniques first are performed on a coarse scale, with the result being used as an input for use of the optical flow techniques at a finer scale. Two or three or several scales may be iterated. In certain embodiments, the scales are related according to increasing number of pixels per grid segment, e.g., each coarser scale has a grid size twice as long at each side as the next finer scale, so as to enclose four times as many pixels per grid segment as does the next finer scale. Denoting the number of scales as S, the maximum measurable velocity for any coordinates now is found as

\vec{ν}ₘₐₓ=2^{S-1}*PRF*Δ\vec{r}.

In case of lower PRF or flow measurements in shallow regions, higher order pyramids could be considered. It is the large number of beams that now can be received simultaneously for ensemble sampling, which has enabled use of pyramidal optical flow.

The coarsest scale is used to identify any regions with flow velocity of about the maximum measurable velocity. Each successively finer scale is used to identify regions with flow velocity of about a binary fraction of the maximum measurable velocity, e.g., about one half, about one quarter, about one eighth, etc. Precision of flow measurement is somewhat dependent upon the precision of pixel intensity, i.e. how many gradations of brightness can be sensed.

In an exemplary embodiment, Lucas-Kanade optical flow technique is used. This technique adds a presumption that the flow in a pixel neighborhood (e.g., a pixel and its surrounding eight pixels; or four adjacent pixels) is constant, and uses that constraint to impose a least squares fit for estimating the flow from brightness variations:

ν=(A^{T}A)⁻¹A^{T}\vec{b}

where

$$A = [ \begin{matrix}{I}_{x} ( {p}_{1} ) , {I}_{y} ( {p}_{1} ) \\ {I}_{x} ( {p}_{2} ) , {I}_{y} ( {p}_{2} ) \\ … \\ {I}_{x} ( {p}_{n} ) , {I}_{y} ( {p}_{n} )\end{matrix} ] ; \vec{b} = [ \begin{matrix}- {I}_{t} ( {p}_{1} ) \\ - {I}_{t} ( {p}_{2} ) \\ … \\ - {I}_{t} ( {p}_{n} )\end{matrix} ] ; \vec{v} = [ \begin{matrix}{V}_{x} \\ {V}_{y}\end{matrix} ] .$$

A test implementation was made on a GPU using Open Computing Language (OpenCL). Tests were run on a high end GPU (AMD FirePro W7000). Reference GPU implementations were made for SAD, SSD and SSD by convolution. While for a certain setup, the SAD performed at 87.7 ms/frame, optical flow provided results within 21.4, 36.8, or 47.2 ms for optical pyramid approach at 1, 2, or 3 scales respectively.

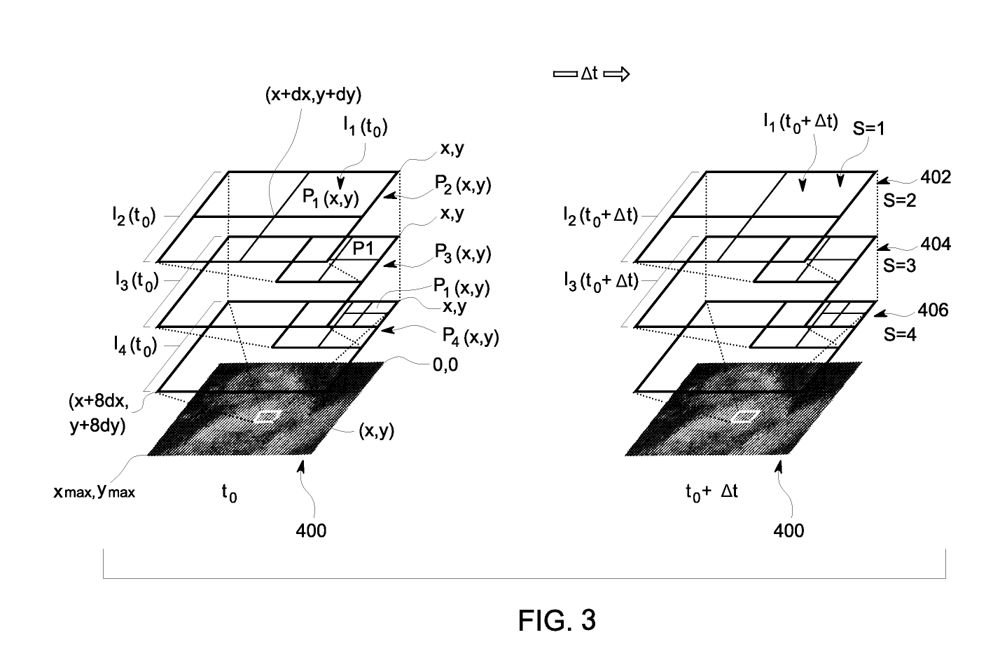

Still referring to FIG. 2, the sampled beams 310 provide data to support sequential Doppler ultrasound images 400, shown in FIG. 3. Referring to the prior discussion of pyramidal optical flow technique, pyramid layers 402, 404, and 406 respectively have scale S=2, S=3, and S=4, with corresponding pixel grid sizes 4, 8, and 16. Intensities I₂, I₃, I₄ respectively are averaged across the different numbers of pixels, and optical flow technique then is applied at each scale to estimate transverse components of flow velocities. The Doppler pixel intensities, themselves, provide for measurement of beamwise (axial/radial) components of flow velocities. Vector summing provides a six dimensional flow field suitable for diagnostics.

Although FIG. 3 shows only two sequential images 400, from which embodiments of the inventive method can produce only a first combined flow field, in certain embodiments the pyramidal optical flow technique is repeatedly applied across consecutive images, e.g., first, second, and third image; or first, second, third, and fourth images; etc., all the way up to the total number of packets 302, 304, 306, 308 within the ensemble 300, thereby providing a plurality of sequential combined flow fields. Thus, sampling more than two consecutive images enables consistency tracking across the plurality of sequential combined flow fields, as well as filtering of the combined flow fields. As one example of filtering, it may typically be expected that so long as a patient maintains a requested resting posture (e.g., prone, supine, reclining, sitting, or standing), blood velocity at any particular anatomic feature will remain consistent with a time-cyclic function driven by the patient's resting pulse in that posture. Thus, for sufficient sample durations (on the order of three or more heartbeats), it can be possible to estimate the time-cyclic function and to filter the accumulated flow fields for outlier values. As another example of filtering, a plurality of combined flow fields can be averaged or can be collapsed to median vectors so as to obtain more robust estimates.

Thus, embodiments of the invention implement a method that includes obtaining first and second ensemble ultrasound image data; computing a coarse transverse flow field by applying optical flow technique to compare the second ensemble ultrasound image data to the first ensemble ultrasound image data at a coarse scale; computing a fine transverse flow field by applying optical flow technique to compare the second ultrasound image data to the first ultrasound image data at a scale that is finer than the coarse scale; and superimposing the fine transverse flow field onto the coarse transverse flow field to form a first combined flow field. The method may also include rendering in real time an image that displays the first combined flow field including single and multi-pixel flow displacements. The method also may include computing and superimposing additional flow fields by comparison of the first and second ultrasound images at additional scales. The first and second ultrasound image data may be compared based on Doppler pixel intensities. The method also may include vector summing the superimposed transverse flow fields with the Doppler pixel intensities to obtain a six dimensional flow field. The first and second ultrasound packets may be consecutive. Lucas-Kanade optical flow technique may be used. In certain embodiments, the method may be implemented entirely within a display processing unit of an ultrasound diagnostic apparatus. The method also may include obtaining third ultrasound image data; computing a second coarse transverse flow field by applying an optical flow technique to compare the third ultrasound image data to the second ultrasound image data at a coarse scale; computing a second fine transverse flow field by applying optical flow technique to compare the second ultrasound image data to the first ultrasound image data at a scale that is finer than the coarse scale; superimposing the second fine transverse flow field onto the second coarse transverse flow field to form a second combined flow field; and filtering the first and second combined flow fields. Filtering may include at least one of averaging, collapsing to median vectors, or eliminating outliers from an estimated time-cyclic function

Other embodiments provide a display processing unit operatively connected to receive ultrasound image data from an ultrasound probe, and configured to obtain first and second ultrasound image data by scanning a target object, compute a coarse transverse flow field by applying optical flow technique to compare the second ultrasound image data to the first ultrasound image data at a coarse scale, compute a fine transverse flow field by applying optical flow technique to compare the second ultrasound image data to the first ultrasound image data at a scale that is finer than the coarse scale, and superimpose the fine transverse flow field onto the coarse transverse flow field. The display processing unit may further be configured to compute and superimpose additional flow fields by comparison of the first and second ultrasound images at additional scales. The first and second ultrasound image data may be compared based on Doppler pixel intensities. The display processing unit also may be configured to vector sum the superimposed transverse flow fields with the Doppler pixel intensities to obtain a six dimensional flow field. The first and second ultrasound image data may be consecutive. Lucas-Kanade optical flow technique may be used.

Yet other embodiments provide an article, which is non-transitory computer readable media encoded with a velocity vector field visualization produced by a process that included obtaining first and second ultrasound image data by scanning a target object; computing a coarse transverse flow field by applying optical flow technique to compare the second ultrasound image data to the first ultrasound image data at a coarse scale; computing a fine transverse flow field by applying optical flow technique to compare the second ultrasound image data to the first ultrasound image data at a scale that is finer than the coarse scale; and superimposing the fine transverse flow field onto the coarse transverse flow field. The image may include additional superimposed flow fields that were obtained by comparison of the first and second ultrasound images at additional scales. The first and second ultrasound image data may be compared based on Doppler pixel intensities. The superimposed transverse flow fields may be summed with the Doppler pixel intensities to obtain a six dimensional flow field. The first and second ultrasound image data may be consecutive. Lucas-Kanade optical flow technique may be used.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, terms such as “first,” “second,” “third,” “upper,” “lower,” “bottom,” “top,” etc. are used merely as labels, and are not intended to impose numerical or positional requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose several embodiments of the invention, including the best mode, and also to enable one of ordinary skill in the art to practice embodiments of the invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal language of the claims.

As used herein, an element or step recited in the singular and proceeded with the word “a” or “an” should be understood as not excluding plural of the elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

Since certain changes may be made in the above-described - - - , without departing from the spirit and scope of the invention herein involved, it is intended that all of the subject matter of the above description or shown in the accompanying drawings shall be interpreted merely as examples illustrating the inventive concept herein and shall not be construed as limiting the invention.

What is claimed is:

1. A method comprising:
obtaining first and second ultrasound image data;
computing a coarse transverse flow field by applying an optical flow technique to compare the second ultrasound image data to the first ultrasound image data at a coarse scale;
computing a fine transverse flow field by applying optical flow technique to compare the second ultrasound image data to the first ultrasound image data at a scale that is finer than the coarse scale; and
superimposing the fine transverse flow field onto the coarse transverse flow field to form a first combined flow field.

2. The method of claim 1, further comprising:
rendering in real time an image that displays the first combined flow field including single and multi-pixel flow displacements.

3. The method of claim 1, further comprising computing and superimposing additional flow fields by comparison of the first and second ultrasound images at additional scales.

4. The method of claim 1, wherein the first and second ultrasound image data are compared based on Doppler pixel intensities.

5. The method of claim 4, further comprising vector summing the superimposed transverse flow fields with the Doppler pixel intensities to obtain a six dimensional flow field.

6. The method of claim 1, wherein the first and second ultrasound image data are consecutive.

7. The method of claim 1, wherein Lucas-Kanade optical flow technique is used.

8. The method of claim 1, being implemented entirely within a display processing unit of an ultrasound diagnostic apparatus.

9. The method of claim 1, further comprising displaying a first image of the superimposed flow fields.

10. The method of claim 1, further comprising:
obtaining third ultrasound image data;
computing a second coarse transverse flow field by applying an optical flow technique to compare the third ultrasound image data to the second ultrasound image data at a coarse scale;
computing a second fine transverse flow field by applying optical flow technique to compare the second ultrasound image data to the first ultrasound image data at a scale that is finer than the coarse scale;
superimposing the second fine transverse flow field onto the second coarse transverse flow field to form a second combined flow field; and
filtering the first and second combined flow fields.

11. The method of claim 10, wherein filtering includes at least one of averaging, collapsing to median vectors, or eliminating outliers from an estimated time-cyclic function.

12. An apparatus comprising:
a display processing unit operatively connected to receive ultrasound image data from an ultrasound probe, and configured to obtain first and second ultrasound image data by scanning a target object; compute a coarse transverse flow field by applying optical flow technique to compare the second ultrasound image data to the first ultrasound image data at a coarse scale; compute a fine transverse flow field by applying optical flow technique to compare the second ultrasound image data to the first ultrasound image data at a scale that is finer than the coarse scale; and superimpose the fine transverse flow field onto the coarse transverse flow field.

13. The apparatus of claim 12, further configured to compute and superimpose additional flow fields by comparison of the first and second ultrasound images at additional scales.

14. The apparatus of claim 12, wherein the first and second ultrasound image data are compared based on Doppler pixel intensities.

15. The apparatus of claim 12, further configured to vector sum the superimposed transverse flow fields with the Doppler pixel intensities to obtain a six dimensional flow field.

16. The apparatus of claim 12, wherein the first and second ultrasound image data are consecutive.

17. The apparatus of claim 12, wherein Lucas-Kanade optical flow technique is used.

18. An article comprising:
non-transitory computer readable media encoded with a velocity vector field visualization produced by a process that includes obtaining first and second ultrasound image data by scanning a target object; computing a coarse transverse flow field by applying optical flow technique to compare the second ultrasound image data to the first ultrasound image data at a coarse scale; computing a fine transverse flow field by applying optical flow technique to compare the second ultrasound image data to the first ultrasound image at a scale that is finer than the coarse scale; and superimposing the fine transverse flow field onto the coarse transverse flow field.

19. The article of claim 18, wherein the image includes additional superimposed flow fields that were obtained by comparison of the first and second ultrasound images at additional scales.

20. The article of claim 18, wherein the first and second ultrasound image data were compared based on Doppler pixel intensities.

21. The article of claim 18, wherein the superimposed transverse flow fields were summed with the Doppler pixel intensities to obtain a six dimensional flow field.
